(19) United States
(12) Patent Application Publication
(10) Pub. No.: US 2022/0218235 A1
(43) Pub. Date: Jul. 14, 2022

(54) DETECTION OF CONDITIONS USING EAR-WEARABLE DEVICES

(71) Applicant: Starkey Laboratories, Inc., Eden Prairie, MN (US)

(72) Inventors: Justin R. Burwinkel, Eden Prairie, MN (US); Kyle Acker, Eden Prairie, MN (US)

(21) Appl. No.: 17/563,762

(22) Filed: Dec. 28, 2021

Related U.S. Application Data
(60) Provisional application No. 63/131,050, filed on Dec. 28, 2020.

(51) Int. Cl. A61B 5/12; A61B 5/00; A61B 5/03

(52) U.S. Cl. CPC A61B 5/12; A61B 2562/0204; A61B 5/036; A61B 5/6817

(57) ABSTRACT

Embodiments herein relate to ear-wearable device that can be used to detect conditions associated with the ears and related methods. In an embodiment, an ear-wearable device is included having a control circuit, a microphone, an electroacoustic transducer for generating sound in electrical communication with the control circuit, a motion sensor, and a power supply circuit, wherein the ear-wearable device is configured to provide auditory stimulation across a range of frequencies with the electroacoustic transducer, detect vibrations within or about the ear with the microphone, and identify a resonant vibrational frequency based on detected vibrations. Other embodiments are also included herein.

This application claims the benefit of U.S. Provisional Application No. 63/131,050, filed Dec. 28, 2020, the content of which is herein incorporated by reference in its entirety.

### FIELD

Embodiments herein relate to ear-wearable device that can be used to detect conditions associated with the ears and related methods.

### BACKGROUND

Fluid in the ear describes a condition including an accumulation of fluid behind the tympanic membrane that can occur under circumstances where fluid drainage from the middle ear is impaired. Causes of fluid in the ear can include both acute and chronic causes including allergies, congestion, bacterial infection, viral infection, enlarged sinus tissue, nasal polyps, inflammation of the tonsils or adenoids, or various growths which can block the Eustachian tube, chemical irritants, barotrauma, craniofacial abnormalities, and the like. Conditions associated with fluid in the ear can include acute otitis media, serous otitis media, otitis media with effusion, chronic otitis media with effusion, and chronic suppurative otitis media, amongst others. Fluid in the ear can reduce movement of the tympanic membrane and middle ear bones, leading to trouble hearing as well as other problems related to proper functioning of the inner ear, amongst other things.

Many other conditions (chronic or acute) associated with the ears can also negatively impact hearing and cause other problems including, but not limited to, large vestibular aqueduct (LVA), enlarged vestibular aqueduct (EVA), semi-circular canal dehiscence, gross bony abnormalities, temporary occlusion, abnormal cerumen deposition, changes in tympanic membrane stiffness, perforations of the tympanic membrane, calcification or scaring of the tympanic membrane, monomeric tympanic membrane, ossicular chain disarticulation or changes in the stiffness of ligament connections between bones in the inner ear, and the like.

### SUMMARY

Embodiments herein relate to ear-wearable device that can be used to detect conditions associated with the ears and related methods. In a first aspect, an ear-wearable device is included having a control circuit, a microphone in electrical communication with the control circuit, an electroacoustic transducer for generating sound in electrical communication with the control circuit, a motion sensor in electrical communication with the control circuit, and a power supply circuit in electrical communication with the control circuit. The ear-wearable device can be configured to provide auditory stimulation across a range of frequencies with the electroacoustic transducer, detect vibrations within or about the ear with the microphone, and identify a resonant vibrational frequency based on detected vibrations.

In a second aspect, in addition to one or more of the preceding or following aspects, or in the alternative to some aspects, the ear-wearable device can be configured to compare the identified resonant vibrational frequency to a baseline value.

In a third aspect, in addition to one or more of the preceding or following aspects, or in the alternative to some aspects, the ear-wearable device can be configured to record the identified resonant vibrational frequency and calculate any changes in the same over time.

In a fourth aspect, in addition to one or more of the preceding or following aspects, or in the alternative to some aspects, the ear-wearable device can be configured to identify fluid in the middle ear space based on the change in the identified resonant vibrational frequency.

In a fifth aspect, in addition to one or more of the preceding or following aspects, or in the alternative to some aspects, the ear-wearable device can be configured to identify a change in fluid in the middle ear space based on the change in the identified resonant vibrational frequency.

In a sixth aspect, in addition to one or more of the preceding or following aspects, or in the alternative to some aspects, the ear-wearable device can be configured to detect a change in inner ear fluid pressure.

In a seventh aspect, in addition to one or more of the preceding or following aspects, or in the alternative to some aspects, the ear-wearable device can be configured to calculate a change in tympanic membrane stiffness based on the change in the identified resonant vibrational frequency.

In an eighth aspect, in addition to one or more of the preceding or following aspects, or in the alternative to some aspects, the ear-wearable device can be configured to calculate a change in stiffness of ligament connections between bones in the inner ear based on the change in the identified resonant vibrational frequency.

In a ninth aspect, in addition to one or more of the preceding or following aspects, or in the alternative to some aspects, the ear-wearable device can be configured to identify a change in the placement position of the ear-wearable device within an ear canal of the device wearer based on the change in the identified resonant vibrational frequency.

In a tenth aspect, in addition to one or more of the preceding or following aspects, or in the alternative to some aspects, the ear-wearable device can be configured to calculate the location of a standing wave within an ear canal of the device wearer.

In an eleventh aspect, in addition to one or more of the preceding or following aspects, or in the alternative to some aspects, the ear-wearable device can be configured to detect a temporary occlusion of an ear canal of the device wearer based on the change in the identified resonant vibrational frequency.

In a twelfth aspect, in addition to one or more of the preceding or following aspects, or in the alternative to some aspects, the ear-wearable device can be configured to detect a cerumen deposition rate in an ear canal of the device wearer based on the change in the identified resonant vibrational frequency.

In a thirteenth aspect, in addition to one or more of the preceding or following aspects, or in the alternative to some aspects, the ear-wearable device can be configured to estimate the size of the vestibular aqueduct based on the detect vibrations within or about the ear.

In a fourteenth aspect, in addition to one or more of the preceding or following aspects, or in the alternative to some aspects, the ear-wearable device can be configured to determine absorbance for a human detectable sound frequency falling within a frequency range of at least one of below 500 Hz, 500 Hz to 4000 Hz, and above 4000 Hz.

In a fifteenth aspect, in addition to one or more of the preceding or following aspects, or in the alternative to some aspects, the ear-wearable device can be configured to identify the presence of LVAS based on the determined absorbance.

In a sixteenth aspect, in addition to one or more of the preceding or following aspects, or in the alternative to some aspects, the ear-wearable device can be configured to detect a third window abnormality.

In a seventeenth aspect, in addition to one or more of the preceding or following aspects, or in the alternative to some aspects, the ear-wearable device can be configured to detect a presence of a semi-circular canal dehiscence.

In an eighteenth aspect, in addition to one or more of the preceding or following aspects, or in the alternative to some aspects, the ear-wearable device can be configured to detect a gross bony abnormality based on the detect vibrations within or about the ear.

In a nineteenth aspect, in addition to one or more of the preceding or following aspects, or in the alternative to some aspects, the ear-wearable device further can include a second microphone, wherein at least one of the microphones is configured to be positioned within the external auditory canal.

In a twentieth aspect, in addition to one or more of the preceding or following aspects, or in the alternative to some aspects, wherein both microphones are configured to be positioned within the ear canal, wherein the microphones are configured to be positioned at two different positions along a lengthwise axis within the external auditory canal.

In a twenty-first aspect, in addition to one or more of the preceding or following aspects, or in the alternative to some aspects, the ear-wearable device can further include a caloric stimulation generator in electrical communication with the control circuit.

In a twenty-second aspect, in addition to one or more of the preceding or following aspects, or in the alternative to some aspects, the ear-wearable device can further include a pair of electrodes in electrical communication with the control circuit and configured to be positioned within the external auditory canal.

In a twenty-third aspect, in addition to one or more of the preceding or following aspects, or in the alternative to some aspects, the ear-wearable device can be configured to identify a resonant vibrational frequency based on detected vibrations in response to the provided auditory stimulation.

In a twenty-fourth aspect, an ear-wearable device is included having a control circuit, a microphone in electrical communication with the control circuit, an electroacoustic transducer for generating sound in electrical communication with the control circuit, a motion sensor in electrical communication with the control circuit, and a power supply circuit in electrical communication with the control circuit. The ear-wearable device can be configured to provide auditory stimulation as a sweep across a range of frequencies and detect vibrations within or about the ear.

In a twenty-fifth aspect, in addition to one or more of the preceding or following aspects, or in the alternative to some aspects, the ear-wearable device can be configured to identify a resonant vibrational frequency based on detected vibrations, and compare the identified resonant vibrational frequency to a baseline value.

In a twenty-sixth aspect, in addition to one or more of the preceding or following aspects, or in the alternative to some aspects, the ear-wearable device can be configured to identify wideband reflectance based on detected vibrations.

In a twenty-seventh aspect, in addition to one or more of the preceding or following aspects, or in the alternative to some aspects, the ear-wearable device can be configured to measure vestibular evoked myogenic potential (VEMP).

In a twenty-eighth aspect, in addition to one or more of the preceding or following aspects, or in the alternative to some aspects, the ear-wearable device can be configured to detect semicircular canal dehiscence (SSCD) based on the measured vestibular evoked myogenic potential.

In a twenty-ninth aspect, in addition to one or more of the preceding or following aspects, or in the alternative to some aspects, the ear-wearable device can be configured to measure cVEMP by measuring evoked responses in the sternocleidomastoid (SCM) muscle.

In a thirtieth aspect, in addition to one or more of the preceding or following aspects, or in the alternative to some aspects, the ear-wearable device can be configured to measure oVEMP by measuring evoked responses in the inferior oblique muscle.

In a thirty-first aspect, in addition to one or more of the preceding or following aspects, or in the alternative to some aspects, the evoked responses of the sternocleidomastoid (SCM) muscle can be detected through movement detected by the motion sensor.

In a thirty-second aspect, an ear-wearable device is included having a control circuit, a microphone in electrical communication with the control circuit, an electroacoustic transducer for generating sound in electrical communication with the control circuit, a motion sensor in electrical communication with the control circuit, and a power supply circuit in electrical communication with the control circuit, wherein the ear-wearable device is configured to provide auditory stimulation and measure evoked responses of the sternocleidomastoid (SCM) muscle.

In a thirty-third aspect, in addition to one or more of the preceding or following aspects, or in the alternative to some aspects, the auditory stimulation can include at least one of click and 250, 500, 750, and 1000 Hz tone burst stimuli.

In a thirty-fourth aspect, in addition to one or more of the preceding or following aspects, or in the alternative to some aspects, the ear-wearable device further configured to measure cervical vestibular evoked myogenic potential (cVEMP) by evaluating the measured evoked responses of the sternocleidomastoid (SCM) muscle.

In a thirty-fifth aspect, in addition to one or more of the preceding or following aspects, or in the alternative to some aspects, the evoked responses of the sternocleidomastoid (SCM) muscle are detected through movement detected by the motion sensor.

In a thirty-sixth aspect, in addition to one or more of the preceding or following aspects, or in the alternative to some aspects, the ear-wearable device can be further configured to detect semicircular canal dehiscence (SSCD) based on the measured vestibular evoked myogenic potential.

In a thirty-seventh aspect, an ear-wearable device is included having a control circuit, a microphone in electrical communication with the control circuit, an electroacoustic transducer for generating sound in electrical communication with the control circuit, a motion sensor in electrical communication with the control circuit, a caloric stimulation generator in electrical communication with the control circuit, and a power supply circuit in electrical communication with the control circuit, wherein the ear-wearable device is configured to deliver caloric stimulation within or about the ear.

In a thirty-eighth aspect, in addition to one or more of the preceding or following aspects, or in the alternative to some aspects, the ear-wearable device can be further configured to detect an evoked response from the caloric stimulation device.

In a thirty-ninth aspect, in addition to one or more of the preceding or following aspects, or in the alternative to some aspects, the evoked response can include a detected movement.

In a fortieth aspect, in addition to one or more of the preceding or following aspects, or in the alternative to some aspects, the detected movement can include at least one of a movement of the head and a movement of the eyes.

In a forty-first aspect, in addition to one or more of the preceding or following aspects, or in the alternative to some aspects, the ear-wearable device can further include a temperature sensor in electrical communication with the control circuit.

In a forty-second aspect, an ear-wearable device is included having a control circuit, a microphone in electrical communication with the control circuit, an electroacoustic transducer for generating sound in electrical communication with the control circuit, a motion sensor in electrical communication with the control circuit, a power supply circuit in electrical communication with the control circuit, and at least one of a caloric stimulation device in electrical communication with the control circuit and an electrical stimulation electrode in electrical communication with the control circuit.

In a forty-third aspect, in addition to one or more of the preceding or following aspects, or in the alternative to some aspects, the ear-wearable device is configured to deliver at least one of caloric stimulation and electrical stimulation within or about the ear.

In a forty-fourth aspect, in addition to one or more of the preceding or following aspects, or in the alternative to some aspects, the ear-wearable device is configured to monitor for an evoked response resulting from the at least one of caloric stimulation and electrical stimulation.

In a forty-fifth aspect, a method of detecting an abnormal ear morphology is included, the method including providing auditory stimulation across a range of frequencies with an ear-wearable device, detecting vibrations within or about the ear with the ear-wearable device, identifying a resonant vibrational frequency based on detected vibrations, and comparing the identified resonant vibrational frequency to a baseline value.

In a forty-sixth aspect, in addition to one or more of the preceding or following aspects, or in the alternative to some aspects, the method can further include evaluating signals from a motion sensor before and/or during the operation of providing auditory stimulation across a range of frequencies.

In a forty-seventh aspect, a method of treating nystagmus with an ear-wearable device is included, the method including detecting an occurrence of nystagmus in a device wearer, providing caloric stimulation to at least one of the anterior canal, horizontal canal, and the posterior canal of an inner ear of the device wearer with an ear-wearable device, monitoring the device wearer for further occurrences of nystagmus.

In a forty-eighth aspect, in addition to one or more of the preceding or following aspects, or in the alternative to some aspects, the caloric stimulation is provided to both the right ear and the left ear simultaneously.

In a forty-ninth aspect, in addition to one or more of the preceding or following aspects, or in the alternative to some aspects, the method can further include monitoring the device wearer for motion during the provision of caloric stimulation.

In a fiftieth aspect, in addition to one or more of the preceding or following aspects, or in the alternative to some aspects, the caloric stimulation is provided using a Joule heating device connected to the ear-wearable device.

In a fifty-first aspect, in addition to one or more of the preceding or following aspects, or in the alternative to some aspects, the caloric stimulation is provided using an infrared device connected to the ear-wearable device.

In a fifty-second aspect, in addition to one or more of the preceding or following aspects, or in the alternative to some aspects, the method can further include prompting the ear-wearable device wearer to be seated or lie down prior to the provision of caloric stimulation.

In a fifty-third aspect, in addition to one or more of the preceding or following aspects, or in the alternative to some aspects, the method can further include querying the ear-wearable device wearer for assent prior to the provision of caloric stimulation.

In a fifty-fourth aspect, in addition to one or more of the preceding or following aspects, or in the alternative to some aspects, the method can further include detecting a sedentary period of the ear-wearable device wearer with a motion sensor prior to the provision of caloric stimulation.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE FIGURES

Aspects may be more completely understood in connection with the following figures (FIGS.), in which:

FIG. 1 is a view of ear anatomy.

FIG. 2 is a schematic view of an ear-wearable device in accordance with various embodiments herein.

FIG. 3 is a schematic view of an ear-wearable device within the ear anatomy in accordance with various embodiments herein.

FIG. 4 is a schematic view of an external auditory canal in accordance with various embodiments herein.

FIG. 5 is a schematic view of an external auditory canal in accordance with various embodiments herein.

FIG. 6 is a schematic view of an external auditory canal in accordance with various embodiments herein.

FIG. 7 is a schematic view of an external auditory canal in accordance with various embodiments herein.

FIG. 8 is a schematic view of an external auditory canal in accordance with various embodiments herein.

FIG. 9 is a schematic view of an external auditory canal in accordance with various embodiments herein.

FIG. 10 is a schematic view of a system in accordance with various embodiments herein.

FIG. 11 is a schematic view of an accessory device in accordance with various embodiments herein.

FIG. 12 is a schematic view of components of an ear-wearable device in accordance with various embodiments herein.

While embodiments are susceptible to various modifications and alternative forms, specifics thereof have been shown by way of example and drawings, and will be described in detail. It should be understood, however, that the scope herein is not limited to the particular aspects described. On the contrary, the intention is to cover modifications, equivalents, and alternatives falling within the spirit and scope herein.

### DETAILED DESCRIPTION

As discussed above, various acute and chronic conditions can lead to trouble hearing as well as other problems including weakness, imbalance, vertigo and the like. Techniques of therapeutic intervention exist for many acute and chronic conditions. However, appropriate therapeutic invention may never be applied or applied properly if such conditions cannot be accurately detected and/or monitored. Testing in a clinical setting can detect various conditions. However, clinical testing is limited to occasions of clinical visits and thus is quite limited and cannot readily detect changes occurring over time.

In various embodiments herein, conditions such as those previously described can be detected using ear-wearable devices. Ear-wearable devices, including, but not limited to hearing assistance devices, are uniquely advantageous for detecting conditions because they are designed to be worn as individuals go about their daily lives wherever they may be and over extended periods of time. Thus, not only can conditions be detected regardless of when they may begin to occur (which can be extremely useful for catching acute conditions as quickly as possible), but the progression and/or remediation of conditions can also be accurately tracked which can provide a much more comprehensive view of an individual's condition so that proper therapeutic interventions can be selected, applied, and/or adjusted.

In some embodiments, ear-wearable devices herein can evaluate properties of sound waves within the external ear canal. For example, in various embodiments herein, an ear-wearable device is included that can be configured to detect vibrations within or about the ear, such as within the external ear canal, with a microphone and identify aspects thereof such as which sounds are reflected and which ones pass into the ear and/or proportions of the same, resonant frequencies, positions (locations) of standing waves, and the like.

In some embodiments, ear-wearable devices can also actively determine such properties. For example, in various embodiments herein, an ear-wearable device is included that can be configured to provide auditory stimulation, such as part of a sweep across a range of frequencies, with the electroacoustic transducer, detect vibrations within or about the ear with a microphone, and identify aspects thereof such as which sounds are reflected and which ones pass into the ear and/or proportions of the same, resonant frequencies, positions of standing waves, and the like. These are properties that can be impacted by conditions of interest such as fluid in the ear. Thus, by analyzing such properties, fluid in the ear (as well as other conditions) can be identified.

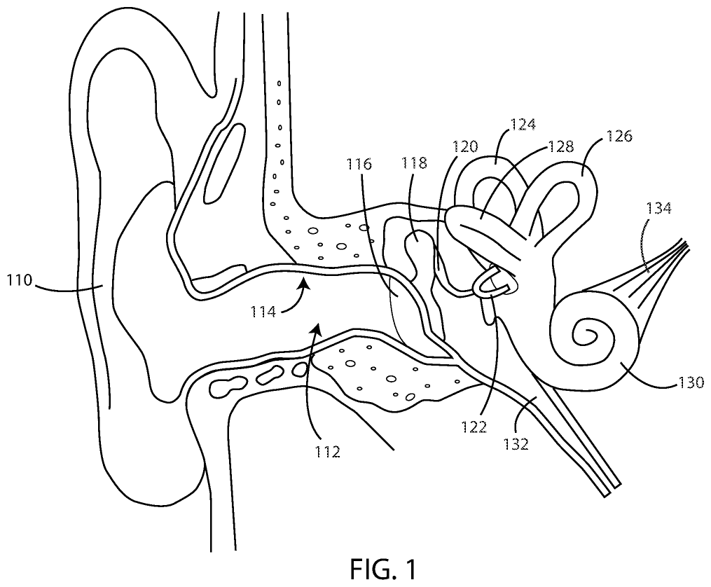

Referring now to FIG. 1, a view of ear anatomy is shown as relevant to embodiments herein. The ear anatomy includes the external ear 110 connecting to the external auditory canal 112 (or ear canal) bordered by wall 114. The adult external auditory canal 112 is divided into an outer cartilaginous portion in its outer third and bony portion in its inner two third. On average, it measures about 2.5 centimeters in length. The posterosuperior wall of the external canal is slightly shorter than the antero-inferior wall because of an antero-inferior inclination of the ear drum. The cartilaginous section of the external auditory canal is angled posterosuperiorly, while the bony canal is inclined anteroinferiorly. These angles give the canal an “S” shape.

Sound waves pass through the external auditory canal 112 and cause the tympanic membrane 116 vibrate. This action moves the tiny chain of auditory bones or ossicle (malleus 118, incus 120, and stapes 122). The stapes 122 contacts the oval window membrane 130 and makes the fluid in the cochlea 130 move. The fluid movement then triggers a response in the auditory nerve 134. A second window (not shown) referred to as the round window acts as a pressure release, equalizing the impedance of the ossicles to the cochlea such that sound waves can be transmitted from one to the other. Normal sound conduction includes transmission through the oval and round windows.

The semicircular canals of the ear include the posterior semicircular canal 124, anterior semicircular canal 126, and the lateral semicircular canal 128. The Eustachian tube 132 (also known as the auditory tube or the pharyngotympanic tube) links the nasopharynx to the middle ear and helps to control pressure within the middle ear generally making it equal with ambient air pressure.

As sound is transmitted along the external auditory canal and through the middle ear, it is reflected wherever there is an impedance mismatch. The most notable such mismatch is between the external auditory canal and the eardrum/middle ear, but there are also others.

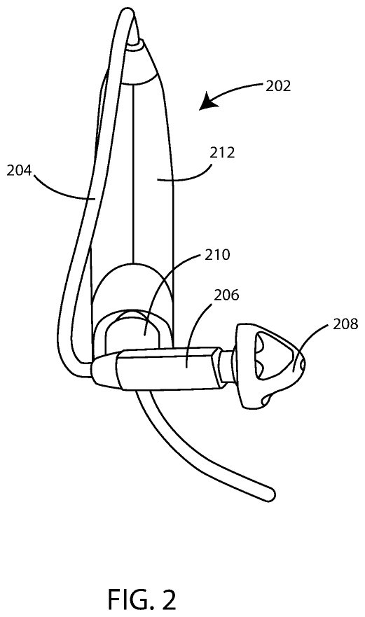

Ear-wearable devices herein can be worn on or in the ears and can measure various properties, such as properties within the external auditory canal 112. Referring now to FIG. 2, a schematic view of an exemplary ear-wearable device 202 is shown in accordance with various embodiments herein. However, it will be appreciated that various other examples of ear-wearable devices are also contemplated herein. The ear-wearable device 202 can include a hearing device housing 212. The hearing device housing 212 can define a battery compartment 210 into which a battery can be disposed to provide power to the device. The ear-wearable device 202 can also include a receiver 206 adjacent to an earbud 208. The receiver 206 an include a component that converts electrical impulses into sound, such as an electroacoustic transducer, speaker, or loudspeaker. A cable 204 or connecting wire can include one or more electrical conductors and provide electrical communication between components inside of the hearing device housing 212 and components inside of the receiver 206. As described further below, various sensors can be mounted on the ear-wearable device such that they can detect properties within the external auditory canal 112.

The ear-wearable device 202 shown in FIG. 2 is a receiver-in-canal type device and thus the receiver is designed to be placed within the ear canal. However, it will be appreciated that many different form factors for ear-wearable devices are contemplated herein. As such, ear-wearable devices herein can include, but are not limited to, behind-the-ear (BTE), in-the ear (ITE), in-the-canal (ITC), invisible-in-canal (ITC), receiver-in-canal (MC), receiver in-the-ear (RITE) and completely-in-the-canal (CIC) type ear-wearable devices. In some embodiments, devices herein can also include cochlear implants and/or osseointegrated devices.

Ear-wearable devices of the present disclosure can incorporate an antenna arrangement coupled to a high-frequency radio, such as a 2.4 GHz radio. The radio can conform to an IEEE 802.11 (e.g., WIFI®) or BLUETOOTH® (e.g., BLE, BLUETOOTH® 4. 2 or 5.0) specification, for example. It is understood that ear-wearable devices of the present disclosure can employ other radios, such as a 900 MHz radio or radios operating at other frequencies or frequency bands. Ear-wearable devices of the present disclosure can be configured to receive streaming audio (e.g., digital audio data or files) from an electronic or digital source and/or play audio from memory. Representative electronic/digital sources (also referred to herein as accessory devices) include an assistive listening system, a TV streamer, a remote microphone, a remote control, a radio, a smartphone, a cell phone/entertainment device (CPED) or other electronic device that serves as a source of digital audio data or files. Systems herein can also include these types of accessory devices as well as other types of devices.

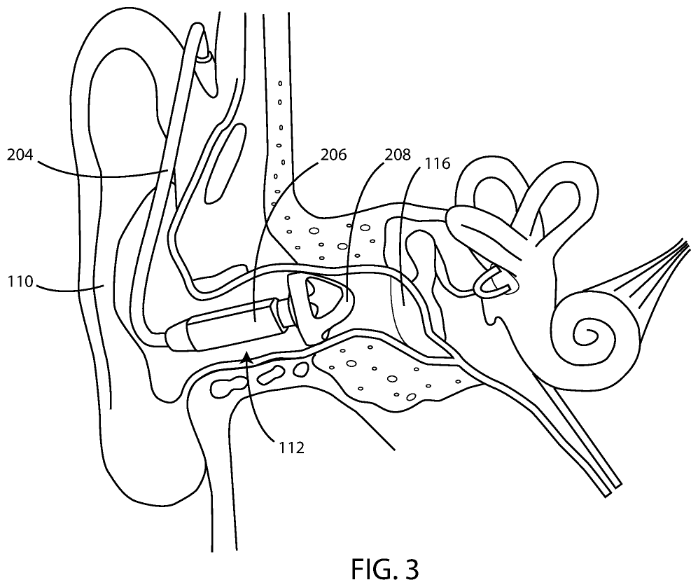

Referring now to FIG. 3, a schematic view of an ear-wearable device 202 within the ear anatomy is shown in accordance with various embodiments herein. FIG. 3 shows components of the ear-wearable device including a cable 204, a receiver 206, and an earbud 208. Sound waves passing through the external ear canal (whether generated by the device itself in an active sensing mode or detected without being generated in a passive sending mode) can reflect off the tympanic membrane and pass back out through external ear canal. Changes in the condition of the ear (acute or chronic) can manifest as changes in properties of such reflection. As such, by monitoring aspects of sound wave reflection and resulting interference conditions, the condition of the ear can also be monitored. Monitoring properties such as resonant vibrational frequencies and/or positions of standing waves can provide useful information regarding the condition of the ear.

In an active sensing mode, the receiver (including, for example, an electroacoustic transducer) and generate sound as a stimulus and then reflected sound can be sensed or otherwise measured such as described in further detail below. The stimulus sound can be at one or more discrete frequencies (such as at 226, 678, and/or 1000 Hz) or can be across a band or bands of frequencies, either simultaneously or as a sweep across frequencies such as at frequencies from the lower bounds of normal human hearing (roughly 20 Hz) up to 2000, 4000, 6000, or 8000 Hz or higher.

In some embodiments, an ear-wearable device herein can also include an external microphone, such as one mounted on the housing or another component. This can be in addition to a microphone positioned with the external auditory canal. In this way, signals from the external microphone can be used to filter out the contribution of ambient sound to the signals of the microphone(s) positioned within the external auditory canal. This can be performed in various ways. In some embodiments, the signals from the external microphone can be subtracted from the signals from the intracanal microphone(s) using analog or digital signal processing techniques. In some embodiments, the signals from the external microphone can be used to normalize the signals from the intracanal microphone(s).

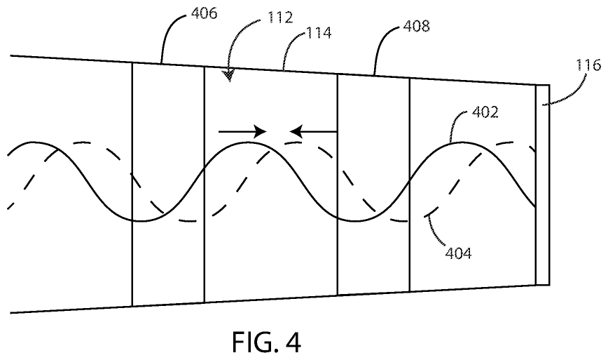

Referring now to FIG. 4, a schematic view of an ear canal is shown in accordance with various embodiments herein. As described above, the external ear canal is typically in an S-shape. For purposes of ease of illustration, the external auditory canal is shown in this view having a first bend 406 (not to scale), which can represent a first zone of curvature. The external auditory canal is also illustrated showing a second bend 408 (not to scale), which can represent a second zone of curvature. Taken together, the curvature of the first zone 406 and second zone 408 results in the “S” shape of the external ear canal, though illustrated herein as being largely straight. The external auditory canal can also be described as divided into a cartilaginous portion (the area where the tissue surrounding the canal is largely cartilage and a bony portion (the area where the tissue surrounding the canal is largely bone), which the bony portion typically beginning beyond the second bend 408.

FIG. 4 also shows an incoming sound wave 402 passing through the external auditory canal 112 and along wall 114. When the incoming sound wave 402 reaches the tympanic membrane 116, a portion of the wave 402 undergoes reflection and is inverted and a portion of the wave undergoes transmission across the tympanic membrane 116 (i.e., admittance). A significant amount of reflection is dependent upon differences in impedance between the external auditory canal 112 and the portions of the ear anatomy including the tympanic membrane 116 and structures beyond the same including the ossicles and the like. FIG. 4 shows a reflected sound wave 404 passing back out through the external auditory canal 112 and along wall 114. In some embodiments, reflected sound waves (or vibrations) can be analyzed. For example, reflected sound waves can be analyzed to detect changes in volumes of reflected sound or sound pressure (which can reflect changes in the tympanic membrane or components of the inner ear), changes in reflected frequencies and/or frequency peaks, changes in reflected frequency bands, other spectral changes, and the like. In some embodiments, reflected sound waves can be analyzed to identify wideband reflectance.

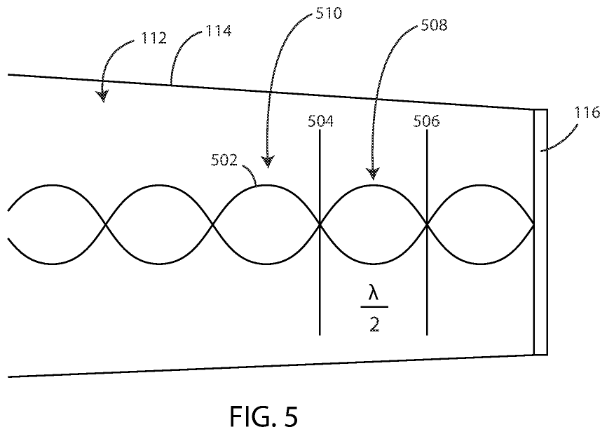

Referring now to FIG. 5, a schematic view of an ear canal is shown in accordance with various embodiments herein. FIG. 5 shows external auditory canal 112 as surrounded by wall 114 along with the tympanic membrane 116. Standing waves (or stationary waves) occur due to the superposition of incoming sounds waves and reflected sound waves. In specific, reflected sound waves can interfere with the incoming sound waves constructively or destructively. Places where the waves interfere destructively and cancel out are known as nodes. Places where the waves interfere constructively resulting in waves of larger amplitude are known as antinodes. FIG. 5 shows a first node 504 and a second node 506, wherein the distance between adjacent nodes is equal to the wavelength divided by 2 (e.g., λ/2). FIG. 5 also shows a first antinode 508 and a second antinode 510. FIG. 5 also shows the peak 502 amplitude of the wave interference pattern resulting in constructive interference at the second antinode 510. Using ear-wearable devices herein and the sensors thereon, the positions of such features within the external ear canal can be detected. This can be used in various ways. For example, by comparing the positions of detected nodes and/or antinodes, and/or changes of the same over time, changes in the condition of the ear can be assessed. In some embodiments, if it is assumed that properties of the ear have not changed, then a change in the position of such features reflects a difference in the placement/position of components of the ear-wearable device and thus changes in the placement/position of the ear-wearable device can be detected.

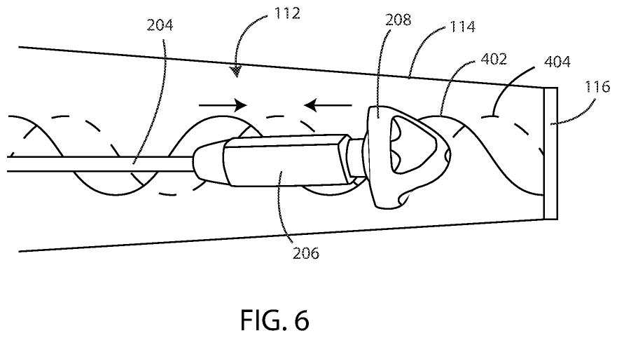

Referring now to FIG. 6, a schematic view of an external auditory canal 112 is shown in accordance with various embodiments herein. Portions of an ear-wearable device are shown including a cable 204 connecting to a receiver 206 and an earbud 208 fitted on the end of the receiver 206. The portions of the ear-wearable device are shown disposed within the external auditory canal 112 surrounded by the wall 114 thereof. FIG. 6 also shows an incoming sound wave 402 and a reflected sound wave 404 that is reflected off the tympanic membrane 116.

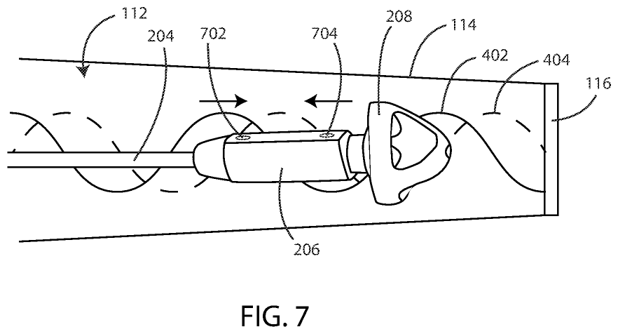

In various embodiments, ear-wearable devices herein can include sensors of various types to detect aspects of sound waves within the external ear canal. Referring now to FIG. 7, a schematic view of an external auditory canal 112 is shown in accordance with various embodiments herein. As before, portions of an ear-wearable device are shown including a cable 204 connecting to a receiver 206 and an earbud 208 fitted on the end of the receiver 206. The portions of the ear-wearable device are shown disposed within the external auditory canal 112 surrounded by the wall 114 thereof. As before, FIG. 7 also shows an incoming sound wave 402 and a reflected sound wave 404 that is reflected off the tympanic membrane 116.

In this particular embodiment, the ear-wearable device also includes a first in-canal microphone 702 and a second in-canal microphone 704. By evaluating the signals of the first in-canal microphone 702 and the second in-canal microphone 704, the ear-wearable device can determine various aspects including the positioning of features such as nodes, antinodes, and the like. It will be appreciated, however, that in various embodiments herein the ear-wearable device may only include a single microphone. In still other embodiments, more than two microphones can be used. Exemplary microphones are described in greater detail below.

In some embodiments, as positioned within the external auditory canal 112, the ear-wearable device 202 can be configured to identify a resonant vibrational frequency. In various embodiments, the ear-wearable device 202 can further be configured to compare the identified resonant vibrational frequency to a baseline value. In various embodiments, the ear-wearable device 202 can further be configured to record the identified resonant vibrational frequency and calculate any changes in the same over time.

In various embodiments, the ear-wearable device 202 can further be configured to identify fluid in the middle ear space based on the change in the identified resonant vibrational frequency. In various embodiments, the ear-wearable device 202 can further be configured to identify a change in fluid in the inner ear based on the change in the identified resonant vibrational frequency, intensity, spectral characteristics, etc.

In various embodiments, the ear-wearable device 202 can further be configured to calculate a change in tympanic membrane stiffness based on the change in the identified resonant vibrational frequencies, frequency bands, spectral patterns, frequency or band intensities, and the like In various embodiments, the ear-wearable device 202 can further be configured to calculate a change in stiffness of ligament connections between bones in the inner ear based on the change in the identified resonant vibrational frequency

In various embodiments, the ear-wearable device 202 can further be configured to identify a change in the placement position of the ear-wearable device 202 within an ear canal of the device wearer based on the change in the identified resonant vibrational frequency

In various embodiments, the ear-wearable device 202 can further be configured to detect a temporary occlusion of an ear canal of the device wearer based on the change in the identified resonant vibrational frequency.

In various embodiments, the ear-wearable device 202 can further be configured to detect a cerumen deposition rate in an ear canal of the device wearer based on the change in the identified resonant vibrational frequency

In various embodiments, the ear-wearable device 202 can be configured to estimate the size of the vestibular aqueduct based on detected vibrations within or about the ear.

In various embodiments, large vestibular aqueduct syndrome (LVAS) can be detected based on the detection of properties associated with the pressure of the inner ear. For example, average absorbance under conditions of ambient pressure at frequencies including 1000, 1189, 1296, 2000 and 4000 Hz can be lower than normal. However, average absorbance under conditions of ambient pressure can be higher than normal at frequencies above 4000 Hz and below 500 Hz. Thus, the device and/or system herein can provide sounds across a range of frequencies and identity LVAS by identifying higher than normal absorbance below 500 Hz and above 4000 Hz and lower than normal absorbance at frequencies between 500 Hz and 4000 Hz.

In various embodiments, the ear-wearable device 202 can be configured to detect a gross bony abnormality based on the detect vibrations within or about the ear.

In some embodiments, a dual receiver system can be used herein. For example, an embodiment of an ear-wearable device including a first receiver (or electroacoustic transducer) can be used to play a sound and a second receiver (or electroacoustic transducer—while not presently being electrically driven) of the ear-wearable device can be used to sense sound, thus temporarily acting as a microphone. As such, in various embodiments herein, a “microphone” can include a receiver or an electroacoustic transducer configured to function as a microphone.

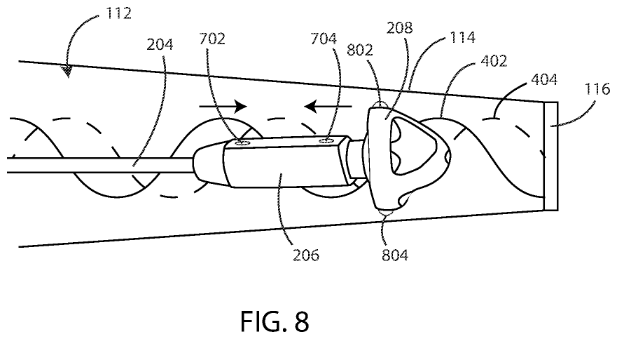

In some embodiments, other aspects can be sensed within the ear canal along with aspects of sound waves therein. Referring now to FIG. 8, a schematic view of an ear canal is shown in accordance with various embodiments herein. Portions of an ear-wearable device are shown including a cable 204 connecting to a receiver 206 and an earbud 208 fitted on the end of the receiver 206. The portions of the ear-wearable device are shown disposed within the external auditory canal 112 surrounded by the wall 114 thereof. FIG. 8 also shows an incoming sound wave 402 and a reflected sound wave 404 that is reflected off the tympanic membrane 116. In this particular embodiment, the ear-wearable device also includes a first in-canal microphone 702 and a second in-canal microphone 704. The ear-wearable device also includes a first electrode 802 and a second electrode 804.

The electrodes can be used for various purposes. In some embodiments, the electrodes can be used for sensing purposes, such as passive sensing of electrical properties. For example, the ear-wearable device can be configured to monitor electrical properties of tissue electrically connecting the first electrode 802 and the second electrode 804 including, but not limited to, electrical potential so that aspects such as muscle fiber recruitment, heart rate and the like can be sensed. In some embodiments, the electrodes can be used as part of an active sensing process. For example, a stimulus can be generated by the ear-wearable device (an auditory stimulus, an electrical stimulus, or the like) and then the electrodes can be used to sense an evoked response. By way of example, vestibular evoked myogenic potential (VEMP) can be measured by stimulating the ear with repetitive sound pulses or click sound stimulation as generated by the ear-wearable device and then measuring an evoked response, such as by measuring surface electromyography (EMG) response using the electrodes. In some embodiments, the auditory stimulation can include at least one of click and 250, 500, 750, and 1000 Hz tone burst stimuli. cVEMP can specifically refer to vestibular evoked myogenic potentials elicited from the sternocleidomastoid muscle. oVEMP can specifically refer to vestibular evoked myogenic potentials elicited from an ocular muscle, such as the inferior oblique muscle.

In some embodiments, the ear-wearable device can be configured to detect semicircular canal dehiscence (SSCD) based on the measured vestibular evoked myogenic potential (VEMP). SSCD is an example of a “third window” type abnormality. As referenced above, normal sound conduction is transmitted through the oval and round windows, which serve as fluid interfaces between air in the middle ear and perilymphatic fluid spaces of the inner ear. However, various conditions can enlarge existing bony channels or create additional defects in the bony labyrinth, producing hydrodynamic third windows. Potential third windows can include bony dehiscence of the semicircular canals, enlargement of the opening of the vestibular aqueduct, dehiscence of the scala vestibuli side of the cochlea, and abnormal bony thinning between the cochlea and vascular channels.

In some embodiments, the electrodes can be used to provide an electrical stimulus (which can be in addition to or instead of a sound stimulus). For example, an electrical stimulus can be applied using the electrodes and then an evoked response can be measured.

It will be appreciated that while FIG. 8 shows the tympanic membrane in an idealized manner and reflection of sound waves off of the same, in some patients, the tympanic membrane may be absent or perforated. However, at least some sound can still reflect back into the external auditory canal and thus embodiments herein are still applicable to such anatomic conditions.

In some embodiments, a stimulus, such as an auditory stimulus can be provided and then a different type of sensor (such as one or more of those described in greater detail below) can be used, instead of or in addition to the electrodes, to detect an evoked response. For example, in some embodiments, evoked responses of the sternocleidomastoid (SCM) muscle can be detected using a sensor other than the electrodes. In some embodiments, evoked responses of the sternocleidomastoid (SCM) muscle are detected through movement detected by the motion sensor.

One aspect of conducting cVEMP testing is the need for the SCM muscle to tense. In various embodiments herein, motion sensors of the system or device(s) can be used to direct the user to assume a head position that will cause the SCM to be tense. Further, in some embodiments, the system or device can provide auditory feedback to assist the device wearer to maintain the tightness. For example, if the system or device detects that the neck is not tight enough, a feedback signal (through audio, video—such as through an accessory device, and/or haptic feedback) can be provided to the device wearer so they are conditioned/encouraged to maintain the proper position that will keep the SCM muscle tense. The system or device can detect that the tightness of the neck based on various inputs such as the position of the head as indicated by a motion sensor or component thereof, inputs from an accessory device, such as a camera thereof, and the like.

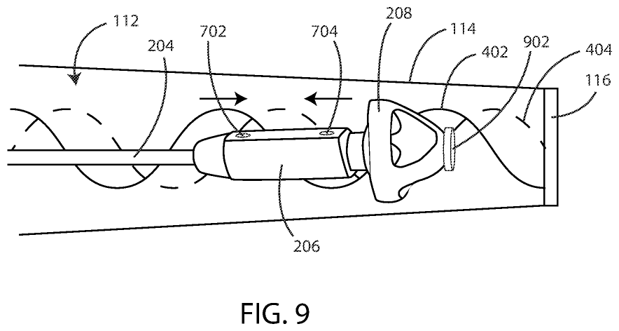

Referring now to FIG. 9, a schematic view of an ear canal is shown in accordance with various embodiments herein. Portions of an ear-wearable device are shown including a cable 204 connecting to a receiver 206 and an earbud 208 fitted on the end of the receiver 206. The portions of the ear-wearable device are shown disposed within the external auditory canal 112 surrounded by the wall 114 thereof. FIG. 9 also shows an incoming sound wave 402 and a reflected sound wave 404 that is reflected off the tympanic membrane 116. In this particular embodiment, the ear-wearable device also includes a first in-canal microphone 702 and a second in-canal microphone 704. In various embodiments, the ear-wearable device can include a caloric stimulation generator 902. The caloric stimulation generator 902 can be in electrical communication with a control circuit of the ear-wearable device. The caloric stimulation generator 902 can be used to deliver caloric stimulation within or about the ear. In various embodiments, the ear-wearable device 202 can be configured to monitor for an evoked response resulting from caloric stimulation and/or electrical stimulation. In some embodiments, the evoked response can include a detected movement, such as that which can be detected with a motion sensor or another sensor herein. Motion can include, for example, various types of motion or movement in response to the stimulus including, but not limited to, movement of a muscle, the head moving, the individual's postural stability changing and the like. In some embodiments, the detected movement can include a movement of the head and/or a movement of the eyes. In some cases, movement of the eyes can be detected using a camera of an accessory device. In some embodiments, the detected movement can include a movement of the head relative to movement of the eyes (e.g., a motion response can be compared to an eye movement response). In some embodiments, an ear-wearable device can also include at least one temperature sensor, which can be used in conjunction with a caloric stimulation generator.

The caloric stimulation generator can be of various types. In some embodiments, the caloric stimulation generator can be a Joule heating device. In some embodiments, the caloric stimulation generator can be an infrared emitting device. In some embodiments, the caloric stimulation generator can be a microwave radiation generator.

In some embodiments, caloric stimulation can cause sudden involuntary movements by the device wearer or feelings of imbalance/vertigo. As such, in some embodiments, the ear-wearable device can be configured to prompt the ear-wearable device wearer to be seated or lie down prior to the provision of caloric stimulation. In some embodiments, the ear-wearable device can be configured to query the ear-wearable device wearer for consent prior to the provision of caloric stimulation. In some embodiments, the ear-wearable device can be configured to detect a sedentary period of the ear-wearable device wearer prior to the provision of caloric stimulation.

Beyond being used to evoke a response, in some embodiments herein caloric stimulation can be used to treat a condition that may result in some vestibular disfunction. For example, benign paroxysmal positional vertigo (BPPV) can be caused by otoliths moving around loose in the inner ear. While not intending to be bound by theory, it is believed that the application of caloric stimulation can speed up the clearance process by which such otoliths are reabsorbed and/or are moved into a place where they no longer cause BPPV. As such, in various embodiments herein, an ear-wearable device can be configured to treat BPPV through caloric stimulation. Similarly, a method herein of treating a vestibular condition such as BPPV can include applying an effective amount of caloric stimulation using components of an ear-wearable device disposed within an external auditory canal of the device wearer.

In various embodiments, an ear-wearable device can be equipped with an auto-vent feature that actively closes off a vent of the ear-wearable device to effectively seal off the ear canal and thereby create greater acoustic and/or thermal separation from the ambient environment during measurements (such as during active sensing measurements). Examples of vent features include, but are not limited to, those found in commonly-owned U.S. patent application Ser. No. 13/720,793 (now issued as U.S. Pat. No. 8,923,543), entitled HEARING ASSISTANCE DEVICE VENT VALVE, and commonly-owned U.S. Provisional Patent Application No. 62/850,805, entitled SOLENOID ACTUATOR IN A HEARING DEVICE, both of which are hereby incorporated by reference herein in their entirety.

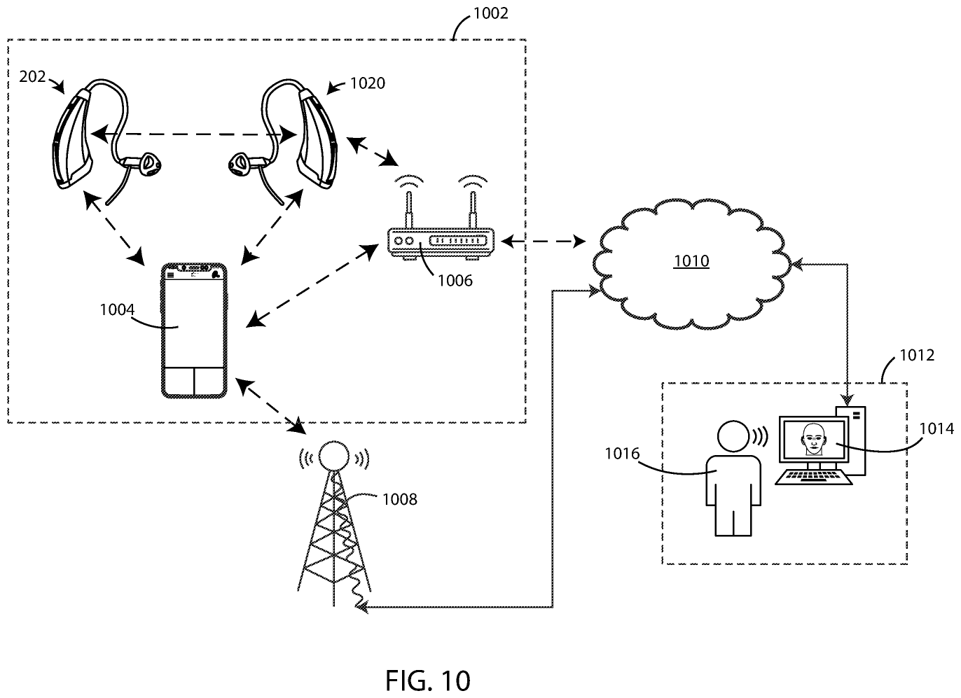

In various embodiments herein, data gathered by one or more ear-wearable devices can be conveyed to a remote location for storage, analysis, and/or presentation to a third party such as a care provider. Referring now to FIG. 10, a schematic view is shown of data and/or signal flow as part of a system in accordance with various embodiments herein. In a first location 1002, a user (not shown) can have a first ear-wearable device 202 and a second ear-wearable device 1020. Each of the ear-wearable devices 202, 1020 can include sensor packages as described herein including, for example, sensors that can be disposed to detect conditions within the external ear canal. The ear-wearable devices 202, 1020 and sensors therein can be disposed on or in opposing ears of the subject. In various embodiments, the ear-wearable devices 202, 1020 and sensors therein can be disposed within opposing ear canals of the subject.

In various embodiments, data and/or signals can be exchanged directly between the first ear-wearable device 202 and the second ear-wearable device 1020. In some embodiments, an external visual display device 1004 with a video display screen, such as a smart phone, can also be disposed within the first location 1002. The external visual display device 1004 can exchange data and/or signals with one or both of the first ear-wearable device 202 and the second ear-wearable device 1020 and/or with an accessory to the ear-wearable devices (e.g., a remote microphone, a remote control, a phone streamer, etc.). The external visual display device 1004 can also exchange data across a data network to the cloud 1010, such as through a wireless signal connecting with a local gateway device, such as a network router 1006 or through a wireless signal connecting with a cell tower 1008 or similar communications tower. In some embodiments, the external visual display device can also connect to a data network to provide communication to the cloud 1010 through a direct wired connection.

In some embodiments, a care provider 1016 (such as an audiologist, physical therapist, a physician or a different type of clinician, specialist, or care provider, or physical trainer) located at a second location 1012 can receive information from devices at the first location 1002 through a data communication network such as that represented by the cloud 1010. The care provider 1016 can use a computing device 1014 to see and interact with the information received. The received information can include, but is not limited to, information regarding detected conditions within the external ear canal of the device wearer along with other health information. In some embodiments, received information can be provided to the care provider 1016 in real time. In some embodiments, received information can be stored and provided to the care provider 1016 at a time point after data has been collected by the ear-wearable devices.

In some embodiments, the care provider 1016 (such as an audiologist, physical therapist, a physician or a different type of clinician, specialist, or care provider, or physical trainer) can send information remotely from the second location 1012 through a data communication network such as that represented by the cloud 1010 to devices at the first location 1002. For example, the care provider 1016 can enter information into the computing device 1014, can use a camera connected to the computing device 1014 and/or can speak into the external computing device. The sent information can include, but is not limited to, instructions/commands for the ear-wearable device(s) and/or instructions for the device wearer.

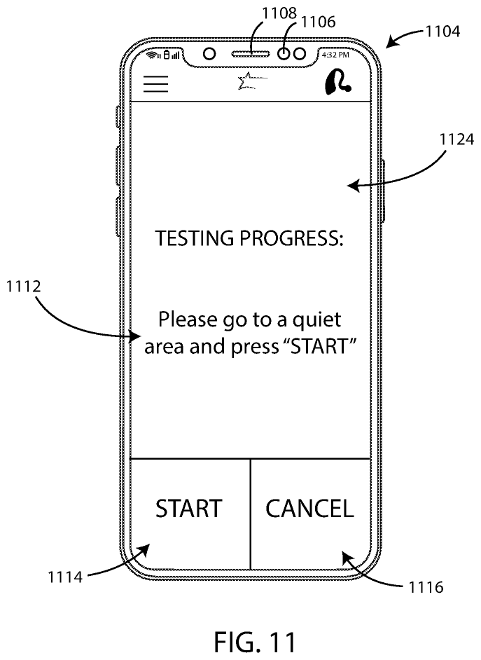

Referring now to FIG. 11, a schematic view of an accessory device is shown in accordance with various embodiments herein. In specific, in this example, the accessory device can take the form of an external visual display device 1104. The external visual display device 1104 can include components such as a camera 1106, and a speaker 1108. The external visual display device 1104 can generate and/or display information for presentation to the device wearer. For example, the external visual display device 1104 can include a display screen 1124 and, in some embodiments, instructions 1112 thereon for the device wearer to follow (or can display other information). For example, to enhance the collection of accurate data in an active sensing mode where sound is generated to test conditions of the external ear canal, the external visual display device 1104 can instruct the device wearer to go to a quiet area. In some embodiments, the external visual display device 1104 can also include user input objects, such as input buttons 1114 and 1116 in order to receive input from the device wearer.

In some embodiments, signals from a motion sensor of a device herein can be evaluated so as to determine whether it is appropriate to execute active or passive sensing or whether the device wearer should be instructed to take some action first. For example, certain types of testing can be benefited by the device wearer being relatively still. If the motion sensor detects motion exceeding a threshold value, then the system or a device thereof can instruct the device wearer to remain still. In some embodiments, the signals from the motion sensor can be used to ensure the device wearer has remained still during the course of a testing procedure. In some embodiments, the motion sensor and/or another sensor of the system can be used to detect the position or posture of the device wearer (either directly or indirectly through a technique similar to dead reckoning). If the device wearer is not in the correct posture for a particular test, then they can be instructed to assume the correct posture. In some embodiments, the device wearer's position or posture can be monitored during the course of a testing procedure.

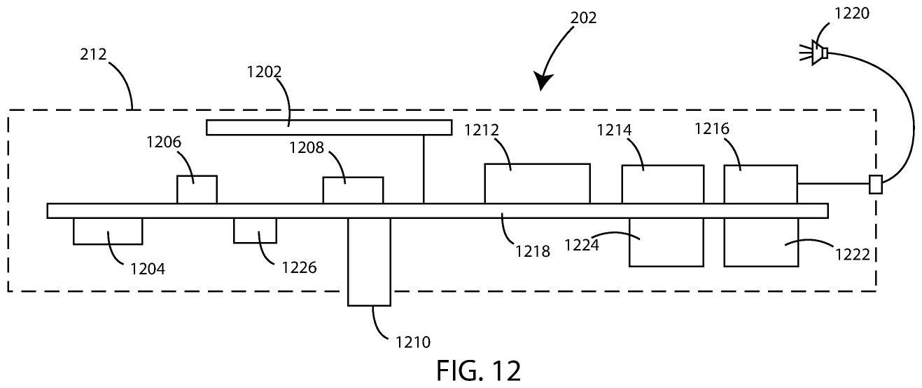

Referring now to FIG. 12, a schematic block diagram is shown of components of an ear-wearable device 202 is shown in accordance with various embodiments herein. The block diagram of FIG. 12 represents a generic ear-wearable device for purposes of illustration. The ear-wearable device 202 shown in FIG. 12 includes several components electrically connected to a flexible mother circuit 1218 (e.g., flexible mother board) which is disposed within housing 212. A power supply circuit 1204 can include a battery and can be electrically connected to the flexible mother circuit 1218 and provides power to the various components of the ear-wearable device 202. One or more microphones 1206 are electrically connected to the flexible mother circuit 1218, which provides electrical communication between the microphones 1206 and a digital signal processor (DSP) 1212. Microphones 1206 can be configured to be external to the auditory canal and/or inside the auditory canal, such as disposed on a component such as the receiver. Among other components, the DSP 1212 incorporates or is coupled to audio signal processing circuitry configured to implement various functions described herein. A sensor package 1214 can be coupled to the DSP 1212 via the flexible mother circuit 1218. The sensor package 1214 can include one or more different specific types of sensors such as those described in greater detail below. One or more user switches 1210 (e.g., on/off, volume, mic directional settings) are electrically coupled to the DSP 1212 via the flexible mother circuit 1218.

An audio output device 1216 is electrically connected to the DSP 1212 via the flexible mother circuit 1218. In some embodiments, the audio output device 1216 comprises a speaker (coupled to an amplifier). In other embodiments, the audio output device 1216 comprises an amplifier coupled to an external receiver 1220 adapted for positioning within an ear of a wearer. The external receiver 1220 can include an electroacoustic transducer, speaker, or loudspeaker. The ear-wearable device 202 may incorporate a communication device 1208 coupled to the flexible mother circuit 1218 and to an antenna 1202 directly or indirectly via the flexible mother circuit 1218. The communication device 1208 can be a BLUETOOTH® transceiver, such as a BLE (BLUETOOTH® low energy) transceiver or other transceiver (e.g., an IEEE 802.11 compliant device). The communication device 1208 can be configured to communicate with one or more external devices, such as those discussed previously, in accordance with various embodiments. In various embodiments, the communication device 1208 can be configured to communicate with an external visual display device such as a smart phone, a video monitor, a video display screen, a smart mirror, a virtual reality device, an augmented reality device, a hologram generator, a tablet, a computer, or the like.

In some embodiments, ear-wearable devices of the present disclosure can incorporate an antenna arrangement coupled to a high-frequency radio, such as a 2.4 GHz radio. The radio can conform to an IEEE 802.11 (e.g., WIFI®) or BLUETOOTH® (e.g., BLE, BLUETOOTH® 4. 2 or 5.0) specification, for example. It is understood that ear-wearable devices of the present disclosure can employ other radios, such as a 900 MHz radio or radios operating at other frequencies or frequency bands. Ear-wearable device of the present disclosure can also include hardware, such as one or more antennas, for NFMI or NFC wireless communications. Ear-wearable devices of the present disclosure can be configured to receive streaming audio (e.g., digital audio data or files) from an electronic or digital source.

Ear-wearable devices of the present disclosure can be configured to receive streaming audio (e.g., digital audio data or files) from an electronic or digital source. Representative electronic/digital sources (also referred to herein as accessory devices) include an assistive listening system, a TV streamer, a radio, a smartphone, a cell phone/entertainment device (CPED) or other electronic device that serves as a source of digital audio data or files. Systems herein can also include these types of accessory devices as well as other types of devices.

In various embodiments, the ear-wearable device 202 can also include a control circuit 1222 and a memory storage device 1224. The control circuit 1222 can be in electrical communication with other components of the device. In some embodiments, a clock circuit 1426 can be in electrical communication with the control circuit. The control circuit 1222 can execute various operations, such as those described herein. The control circuit 1222 can include various components including, but not limited to, a microprocessor, a microcontroller, an FPGA (field-programmable gate array) processing device, an ASIC (application specific integrated circuit), or the like. The memory storage device 1224 can include both volatile and non-volatile memory. The memory storage device 1224 can include ROM, RAM, flash memory, EEPROM, SSD devices, NAND chips, and the like. The memory storage device 1224 can be used to store data from sensors as described herein and/or processed data generated using data from sensors as described herein, including, but not limited to, information regarding exercise regimens, performance of the same, visual feedback regarding exercises, and the like.

### Methods

Many different methods are contemplated herein, including, but not limited to, methods of making, methods of using, and the like. Aspects of system/device operation described elsewhere herein can be performed as operations of one or more methods in accordance with various embodiments herein.

In an embodiment, a method of detecting an abnormal ear morphology is included, the method providing auditory stimulation across a range of frequencies, detecting vibrations within or about the ear, identifying a resonant vibrational frequency based on detected vibrations, and comparing the identified resonant vibrational frequency to a baseline value.

In an embodiment, a method of treating nystagmus with an ear-wearable device is included, the method detecting an occurrence of nystagmus in a device wearer, providing caloric stimulation to at least one of the anterior canal, horizontal canal, and the posterior canal of an inner ear of the device wearer with an ear-wearable device, monitoring the device wearer for further occurrences of nystagmus.

In an embodiment of the method, the caloric stimulation is provided to both the right ear and the left ear simultaneously.

In an embodiment, the method can further include monitoring the device wearer for motion during the provision of caloric stimulation.

In an embodiment of the method, the caloric stimulation is provided using a Joule heating device connected to the ear-wearable device.

In an embodiment of the method, the caloric stimulation is provided using an infrared device connected to the ear-wearable device.

In an embodiment, the method can further include prompting the ear-wearable device wearer to be seated or lie down prior to the provision of caloric stimulation. In some embodiments, the method can further include prompting the device wearer to lie down in a certain position, such as on their side, supine, prone, etc.

In an embodiment, the method can further include querying the ear-wearable device wearer for assent prior to the provision of caloric stimulation.

In an embodiment, the method can further include detecting a sedentary period of the ear-wearable device wearer prior to the provision of caloric stimulation.

### Sensors

Ear-wearable devices herein can include one or more sensor packages (including one or more discrete or integrated sensors) to provide data. The sensor package can comprise one or a multiplicity of sensors. In some embodiments, the sensor packages can include one or more motion sensors (or movement sensors) amongst other types of sensors. Motion sensors herein can include inertial measurement units (IMU), accelerometers, gyroscopes, barometers, altimeters, and the like. The IMU can be of a type disclosed in commonly owned U.S. Pat. No. 9,848,273, which is incorporated herein by reference. In some embodiments, electromagnetic communication radios or electromagnetic field sensors (e.g., telecoil, NFMI, TMR, GMR, etc.) sensors may be used to detect motion or changes in position as well as the individual's location and/or environment. In various embodiments, the sensor package can include a magnetometer. In some embodiments, biometric sensors may be used to detect body motions or physical activity as well as contextual information. Motions sensors can be used to track movement of a patient in accordance with various embodiments herein.

In some embodiments, the motion sensors can be disposed in a fixed position with respect to the head of a patient, such as worn on or near the head or ears. In some embodiments, the operatively connected motion sensors can be worn on or near another part of the body such as on a wrist, arm, or leg of the patient.

According to various embodiments, the sensor package can include one or more of an IMU, and accelerometer (3, 6, or 9 axis), a gyroscope, a barometer, an altimeter, a magnetometer, a magnetic sensor, an eye movement sensor, a pressure sensor, an acoustic sensor, a telecoil, a heart rate sensor, a global positioning system (GPS), a microphone, an acoustic sensor, a wireless radio antenna, an air quality sensor, an optical sensor, a light sensor, an image sensor, a temperature sensor, a physiological sensor such as a blood pressure sensor, an oxygen saturation sensor, a blood glucose sensor (optical or otherwise), a galvanic skin response sensor, a cortisol level sensor (optical or otherwise), an electrocardiogram (ECG) sensor, electroencephalography (EEG) sensor which can be a neurological sensor, eye movement sensor (e.g., electrooculogram (EOG) sensor), myographic potential electrode sensor (EMG), a heart rate monitor, a pulse oximeter or oxygen saturation sensor (SpO2), blood perfusion sensor, hydrometer, sweat sensor, cerumen sensor, pupillometry sensor, hematocrit sensor, or the like.

In some embodiments, the sensor package can be part of an ear-wearable device. However, in some embodiments, the sensor packages can include one or more additional sensors that are external to an ear-wearable device. For example, various of the sensors described above can be part of a wrist-worn or ankle-worn sensor package, or a sensor package supported by a chest strap. In some embodiments, sensors herein can be disposable sensors that are adhered to the device wearer (“adhesive sensors”) and that provide data to the ear-wearable device or another component of the system.

Data produced by the sensor(s) of the sensor package can be operated on by a processor of the device or system.

As used herein the term “inertial measurement unit” or “IMU” shall refer to an electronic device that can generate signals related to a body's specific force and/or angular rate. IMUs herein can include one or more accelerometers (3, 6, or 9 axis) to detect linear acceleration and a gyroscope to detect rotational acceleration and/or velocity. In some embodiments, an IMU can also include a magnetometer to detect a magnetic field.

The eye movement sensor may be, for example, an electrooculographic (EOG) sensor, such as an EOG sensor disclosed in commonly owned U.S. Pat. No. 9,167,356, which is incorporated herein by reference. The pressure sensor can be, for example, a MEMS-based pressure sensor, a piezo-resistive pressure sensor, a flexion sensor, a strain sensor, a diaphragm-type sensor and the like.

The temperature sensor can be, for example, a thermistor (thermally sensitive resistor), a resistance temperature detector, a thermocouple, a semiconductor-based sensor, an infrared sensor, or the like.

The blood pressure sensor can be, for example, a pressure sensor. The heart rate sensor can be, for example, an electrical signal sensor, an acoustic sensor, a pressure sensor, an infrared sensor, an optical sensor, or the like.

The oxygen saturation sensor (such as a blood oximetry sensor) can be, for example, an optical sensor, an infrared sensor, a visible light sensor, or the like.

The electrical signal sensor can include two or more electrodes and can include circuitry to sense and record electrical signals including sensed electrical potentials and the magnitude thereof (according to Ohm's law where V=IR) as well as measure impedance from an applied electrical potential.

It will be appreciated that the sensor package can include one or more sensors that are external to the ear-wearable device. In addition to the external sensors discussed hereinabove, the sensor package can comprise a network of body sensors (such as those listed above) that sense movement of a multiplicity of body parts (e.g., arms, legs, torso).

It should be noted that, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

As used herein, the recitation of numerical ranges by endpoints shall include all numbers subsumed within that range (e.g., 2 to 8 includes 2.1, 2.8, 5.3, 7, etc.).

The headings used herein are provided for consistency with suggestions under 37 CFR 1.77 or otherwise to provide organizational cues. These headings shall not be viewed to limit or characterize the invention(s) set out in any claims that may issue from this disclosure. As an example, although the headings refer to a “Field,” such claims should not be limited by the language chosen under this heading to describe the so-called technical field. Further, a description of a technology in the “Background” is not an admission that technology is prior art to any invention(s) in this disclosure. Neither is the “Summary” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

1. An ear-wearable device comprising:
a control circuit;
a microphone in electrical communication with the control circuit;
an electroacoustic transducer for generating sound in electrical communication with the control circuit;
a motion sensor in electrical communication with the control circuit;
a power supply circuit in electrical communication with the control circuit;
wherein the ear-wearable device is configured to
provide auditory stimulation across a range of frequencies with the electroacoustic transducer;
detect vibrations within or about the ear with the microphone; and
identify a resonant vibrational frequency based on detected vibrations.

2. (canceled)

3. The ear-wearable device of claim 1, the ear-wearable device further configured to record the identified resonant vibrational frequency and calculate any changes in the same over time.

4. The ear-wearable device of claim 3, the ear-wearable device configured to identify fluid in the middle ear space based on the change in the identified resonant vibrational frequency.

5. The ear-wearable device of claim 4, the ear-wearable device configured to identify a change in fluid in the middle ear space based on the change in the identified resonant vibrational frequency.

6. The ear-wearable device of claim 1, the ear-wearable device configured to detect a change in inner ear fluid pressure.

7. The ear-wearable device of claim 3, the ear-wearable device configured to perform at least one of
calculate a change in tympanic membrane stiffness based on the change in the identified resonant vibrational frequency;
calculate a change in stiffness of ligament connections between bones in the inner ear based on the change in the identified resonant vibrational frequency; or
identify a change in the placement position of the ear-wearable device within an ear canal of the device wearer based on the change in the identified resonant vibrational frequency.

8-9. (canceled)

10. The ear-wearable device of claim 3, the ear-wearable device configured to calculate the location of a standing wave within an ear canal of the device wearer.

11. The ear-wearable device of claim 3, the ear-wearable device configured to detect a temporary occlusion of an ear canal of the device wearer based on the change in the identified resonant vibrational frequency.

12. (canceled)

13. The ear-wearable device of claim 1, the ear-wearable device configured to estimate the size of the vestibular aqueduct based on the detect vibrations within or about the ear.

14. The ear-wearable device of claim 1, configured to determine absorbance for a human detectable sound frequency falling within a frequency range of at least one of below 500 Hz, 500 Hz to 4000 Hz, and above 4000 Hz.

15. The ear-wearable device of claim 14, configured to identify the presence of LVAS based on the determined absorbance.

16. The ear-wearable device of claim 1, the ear-wearable device configured to detect a third window abnormality.

17. The ear-wearable device of claim 1, the ear-wearable device configured to detect a presence of a semi-circular canal dehiscence.

18. The ear-wearable device of claim 1, the ear-wearable device configured to detect a gross bony abnormality based on the detect vibrations within or about the ear.

19. The ear-wearable device of claim 1, the ear-wearable device further comprising a second microphone, wherein at least one of the microphones is configured to be positioned within the external auditory canal.

20. The ear-wearable device of claim 1, wherein both microphones are configured to be positioned within the ear canal, wherein the microphones are configured to be positioned at two different positions along a lengthwise axis within the external auditory canal.

21-23. (canceled)

24. An ear-wearable device comprising:
a control circuit;
a microphone in electrical communication with the control circuit;
an electroacoustic transducer for generating sound in electrical communication with the control circuit;
a motion sensor in electrical communication with the control circuit;
a power supply circuit in electrical communication with the control circuit;
wherein the ear-wearable device is configured to
provide auditory stimulation as a sweep across a range of frequencies; and
detect vibrations within or about the ear.

25. The ear-wearable device of claim 24, further configured to identify a resonant vibrational frequency based on detected vibrations; and
compare the identified resonant vibrational frequency to a baseline value.

26-31. (canceled)

32. An ear-wearable device comprising:
a control circuit;
a microphone in electrical communication with the control circuit;
an electroacoustic transducer for generating sound in electrical communication with the control circuit;
a motion sensor in electrical communication with the control circuit;
a power supply circuit in electrical communication with the control circuit;
wherein the ear-wearable device is configured to
provide auditory stimulation; and
measure evoked responses of the sternocleidomastoid (SCM) muscle.

33. The ear-wearable device of claim 32, the auditory stimulation comprising at least one of click and 250, 500, 750, and 1000 Hz tone burst stimuli.

34-54. (canceled)
